(19) United States
(12) Patent Application Publication
(10) Pub. No.: US 2020/0037878 A1
(43) Pub. Date: Feb. 6, 2020

(54) METHOD AND SYSTEM RELATED TO COLLECTION AND CORRELATION OF DATA FROM MULTIPLE SOURCES

(71) Applicant: Nextern, Inc., White Bear Lake, MN (US)

(72) Inventors: Ryan Douglas, Stillwater, MN (US); David Bontrager, Minneapolis, MN (US)

(21) Appl. No.: 16/530,548

(22) Filed: Aug. 2, 2019

Related U.S. Application Data
(60) Provisional application No. 62/714,005, filed on Aug. 2, 2018.

(51) Int. Cl. A61B 5/00; G16H 10/60

(52) U.S. Cl. CPC A61B 5/0006; G16H 10/60; A61B 5/4211; A61B 5/4233; A61B 5/0022

(57) ABSTRACT

Apparatus and methods are directed to virtually verifying authenticity of medical information transmitted from at least one unvalidated communication device associated with a patient being monitored by at least on validated medical device. In an illustrative example, medical information related to patient data may be transmitted through a network for storage in a central database from one or more validated medical devices and one or more unvalidated personal communication devices. The transmitted information may include unique identifiers that enables the patient data to be verified and collated across the validated and unvalidated devices and assigned to a specific data record for a given patient or group of patients.

### CROSS-REFERENCE TO RELATED APPLICATIONS

This application claims the benefit of U.S. Provisional Application Ser. No. 62/714,005, titled “Method and System Related to Collection and Correlation of Data From Multiple Sources,” filed by Ryan Douglas, et al., on Aug. 2, 2018, the entire contents of which are incorporated herein by reference.

### TECHNICAL FIELD

Various embodiments relate generally to verifying and validating electronic health records and, more specifically, to a method and system of virtually correlating, verifying and validating patient medical information transmitted from unvalidated and validated devices.

### BRIEF DESCRIPTION OF THE DRAWINGS

FIG. 1 depicts an illustration of an exemplary system related to data collection and correlation from different sources.

FIG. 2 depicts a flow chart illustrating an exemplary method related to data collection and correlation from different sources.

Like reference symbols in the various drawings indicate like elements.

### DETAILED DESCRIPTION OF ILLUSTRATIVE EMBODIMENTS

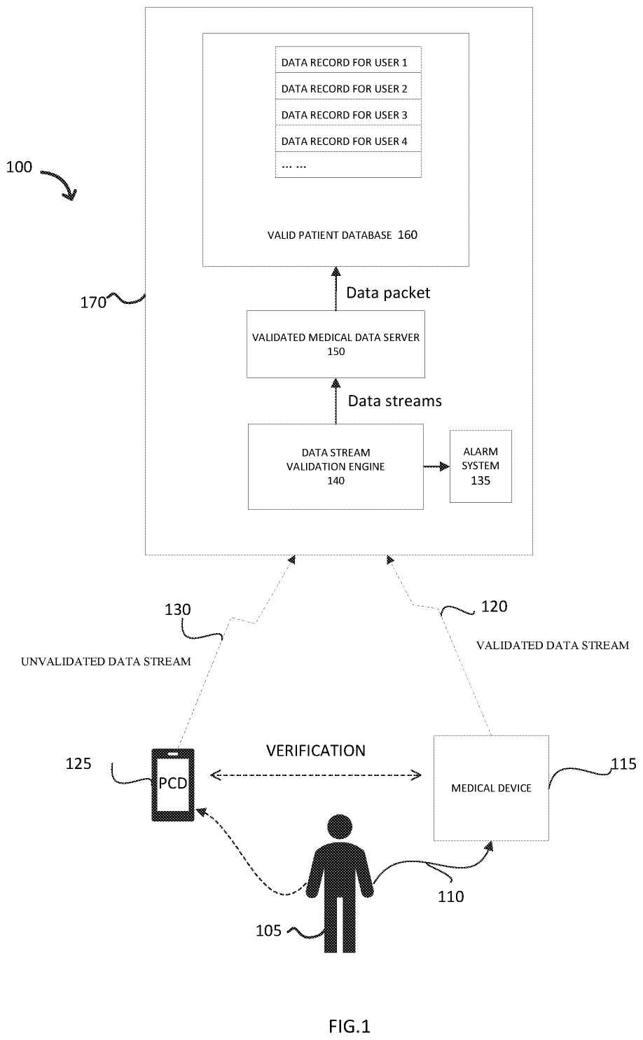

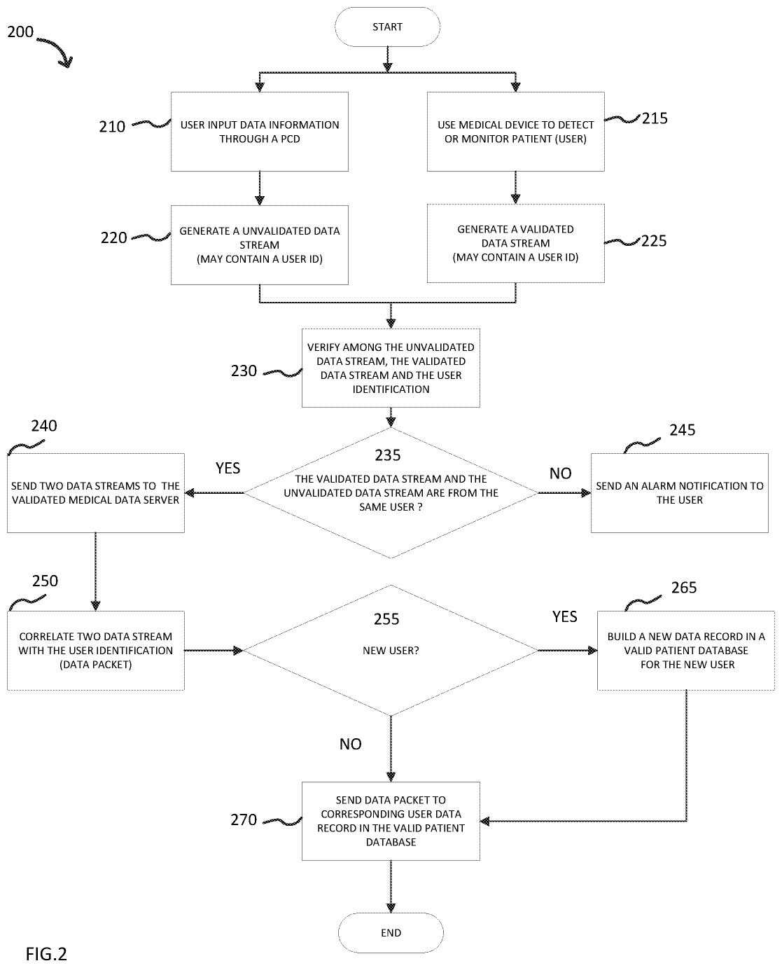

To aid understanding, this document is organized as follows. First, an exemplary system related to collection and correlation of data from multiple sources is briefly introduced with reference to FIG. 1. Second, with reference to FIG. 2, the discussion turns to exemplary embodiments that illustrate an exemplary method related to collection and correlation of data from multiple sources.

FIG. 1 depicts an illustration of an exemplary system related to collection and correlation of data from multiple sources.

In an exemplary system, the system 100 includes a user 105, a medical device 115, and a personal communication device 125 (PCD). The medical device 115, may be validated by respective regulatory department, connected to the user 105 through a monitor link 110 to detect health status related information of the user. The medical device 115 includes but is not limited to an electrocardiograph (ECG), insulin measurement for diabetics, electroencephalograph (EEG) to, for example, detect strokes or epileptic fits, or measurement of galvanic skin response or other correlates to emotional response or mental load as tracked for various psychological or neurological studies. In some implementations, one or more medical devices with a pH sensor can be used to monitor, for example, parameters such as the acidity of a patient's esophagus during diagnosing and treating gastroesophageal reflux disease (GERD). Data obtained by the medical device 115 may form a validated data stream 120.

The PCD 125, by way of example but not limitation, is a hand-held phone, tablet, or a smart watch. The PCD 125 may be installed with a “User Input” application. The application might prompt the user 105 to, for example, respond to survey questions, rate their level of pain or another perceivable status such as their feeling of physical and/or emotional wellbeing, and/or psychological evaluation. The user 105 may also input photos, videos, or audio files recorded by the user, or values the user 105 enters based on external measurements such as recording patient weight as measured by the user's bathroom scale and read by the user 105. Information inputted by the user 105 via the PCD 125 may form an unvalidated data stream 130. Both the validated data stream 120 and the unvalidated data stream 130 may contain a time stamp, and either of the data streams 120, 130 may contain authentication information, such as, for example, user identification information.

The system 100 also includes a central location 170. The central location 170 includes a data stream validation engine 140, an alarm system 135, a validated medical data server 150 and a valid patient database 160. The engine 140 may be configured to verify whether the validated data stream 120 and the unvalidated data stream 130 are associated with the same user 105. The verification that the engine 140 used may include but is not limited to, temporary challenge code, rolling code, or email/text code. After comparing the time stamp and verifying user identification information, if the validated data stream or the unvalidated data stream are not from the same user 105, the engine would send a signal to the alarm system 135, the alarm system then may send a notification to the user 105. If both two data streams 120, 130 are from the same user 105, then the engine 140 would send the two data streams 120, 130 to the validated medical data server 150, and the validated medical data server 150 would then correlate the two data streams 120, 130 with the user 105. Accordingly, a data packet including the two independent data streams 120, 130 and the user identification are formed.

If the user 105 is an existing user, the data packet would be sent directly into corresponding user data record in the valid patient database 160, according to the user identification accompanying each data packet. If the user 105 is a new user, then validated medical data server 150 would build a new data record in the valid patient database 160, and the medical data server 150 then send the data packet into the corresponding new data record specified for this new user.

A number of implementations have been described. Nevertheless, it will be understood that various modification may be made. For example, advantageous results may be achieved if the steps of the disclosed techniques were performed in a different sequence, or if components of the disclosed systems were combined in a different manner, or if the components were supplemented with other components.

In one exemplary aspect, a data collection and correlation system 100 includes a user 105 and a medical device 115. The medical device 115 is connected to the user 105 through a monitor link 110 to obtain a first data stream 120. The system also includes a PCD 125, which can obtain a second data stream 130 from the user 105. The system also includes a central location 170. The central location 170 collects and correlates the first data stream 120 and the second data stream 130, the central location 170 includes a data stream validation engine 140, an alarm system 135, a validated medical data server 150 correlating the first data stream, the second data stream and the user 105 and a valid patient database 160 storing correlated data record. The first data stream 120 and the second data stream 130 are independent, the data stream validation engine 140 then may make a decision based on the verification result of the first data stream 120, the second data stream 130 and the user 105. Within the database, data records may or may not be anonymized and may or may not comply with specific research standards and/or regulations such as HIPAA.

FIG. 2 depicts a flow chart illustrating an exemplary method related to data collection and correlation from different sources. Method 200 includes: Step 210, a user inputs data information through a PCD and Step 215, uses one or more medical devices to detect or monitor patient. Then, Step 220, PCD may generate a unvalidated data stream and Step 225, medical devices may generate a validated data stream. Step 230 and Step 235, data stream validation engine then may verify among the unvalidated data stream, the validated data stream and the user to see whether the two data streams are from the same user. If the two data streams are not from the same user, Step 245, the engine may then send a notification to the user.

Although various embodiments have been described with reference to the Figures, other embodiments are possible. For example, in some embodiments, the system 100 may be used to collect and correlate data related to gastroesophageal reflux disease (GERD). GERD is a condition in which acid from the stomach leaks into the esophagus. Medical devices with a pH sensor are used to monitor the acidity of a patient's esophagus in the course of diagnosing and treating GERD. In existing treatments, the sensor is introduced to the patient's esophagus by passing a catheter terminated with a pH sensor through the nose down into the esophagus.

In some embodiments, the medical device 115 such as a GERD-related pH sensor may send a packet of data to a nearby personal computing device 125, for example, the patient's smartphone. The smartphone may be installed with a “Data Pass Through” application. The data packet would include identifying information such as current time and date and information to identify the hardware associated with the medical device 115 and/or smartphone. The patient's phone may then transmit the data to a central location 170 or database such as a private or public server accessible through Wi-Fi, mobile data networks, Bluetooth, or other forms of connection. The phone would perform no alteration to the received data, acting only as a transmission station through which the sensor could send the data to, for example, the cloud database. The exemplary system's phone may be installed with a second “User Input” application. The application might prompt the user to, for example, respond to survey questions, rate their level of pain or another perceivable status such as their feeling of physical and or emotional wellbeing. The User Input application might then send the user input as a data packet including identifying information such as time and date stamp, hardware identification of the phone and/or medical device, and a unique patient ID. In some embodiments, the medical device 115 and/or phone may also report location data.

An alternative embodiment may have a single application for the personal computing platform instead of separate “Data Pass Through” and “User Input” applications. An alternative embodiment may use more than two applications to separate functionality in a manner that satisfies various usability, safety, or regulatory requirements.

In an alternative embodiment, the medical device 115, may connect directly to the database without the aid of the PCD 125, being installed with, for example, GSM or other cellular data technology, Wi-Fi, Bluetooth, Bluetooth Low Energy (BLE), or wired communication connections such as USB, Ethernet, fiber optics, RS232, or other novel or established communication architectures. In an alternative embodiment, the medical device 115 may connect directly to the database through the PCD 125 over protocols such as Wi-Fi or Bluetooth, wherein the phone acts as a server or “hotspot” in the chain of network connections between the medical device and the database. Near-range communication technology such as BLE advantageously extends the medical device's battery life compared to technologies with higher power consumption may also be used.

Some embodiments may allow use of multiple medical devices 115 transmitting their respective data. Some embodiments may allow use of multiple PCDs 125. This could improve convenience for the patient if, for example, they do not have a smartphone and are able to use a first personal computer at their workplace and a second personal computer at their home. This could also allow for collection and correlation of data from multiple individuals to provide a data set targeted at analyzing data across a group of people. For example, a group of individuals might each report their feeling of emotional or physical wellbeing or rate their feelings towards other members of the group (if some or all members have shared interactions) or to some stimulus with or without data transmitted from one or more medical devices. With their input data in a centralized database, researchers might analyze group dynamics or differences in individual responses to stimuli received by all or a subset of the group.

Data record of the user may be accessed by researchers or clinicians to aid in diagnosis and treatments. Data in the valid patient database 160 may also be used to correlate patient outcomes to treatments of a single patient or across multiple patients. This could give convenient access to large amounts of data for statistical analysis determining efficacy of existing treatments to direct research into new treatments or to direct treatment recommendations provided by clinicians. Systems may also gather data that is relevant to public health professionals, the collection of which might, for example, allow epidemiological studies of infectious disease, health risk factors, geographical analysis of data (GIS), or other relevant studies that may be performed.

A patient's doctor may receive data, for example, via email, downloaded to a doctor application manually or automatically, or with 3^{rd} party tools that allow controlled access to the database 160. All data may be sent to a single doctor. With different kinds of data generated by a patient, all data or different subsets of that data may be sent to multiple doctors, for example, ECG data to a cardiologist and psychological evaluation data to a mental health professional.

A doctor or other health professional and/or the patient may receive alerts via, for example, email or text message if a patient's data indicates dangerous conditions. This might include heart palpitations measured by a patient's ECG, or suicidal tendencies and other risk factors reported by the patient. The medical device 115 itself may generate the alert and send the alert directly to a medical professional or to the database which in turn would alert the medical professional. The patient's PCD 125 may also generate the alert to send to the database 160 or to other targeted recipients such as a medical professional. Certain medical devices in this system may advantageously have the ability to contact an emergency number such as 911 and provide patient identity and location information to request assistance. Alerts might also be provided to the patient in the form of text, email, or visual or audio feedback on the device itself.

Some aspects of embodiments may be implemented as a computer system. For example, various implementations may include digital and/or analog circuitry, computer hardware, firmware, software, or combinations thereof. Apparatus elements can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Some embodiments may be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example and not limitation, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and, CD-ROM and DVD-ROM disks. The processor and the memory can be supplemented by, or incorporated in, ASICs (application-specific integrated circuits). In some embodiments, the processor and the member can be supplemented by, or incorporated in hardware programmable devices, such as FPGAs, for example.

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. An exemplary embodiment may be implemented in a computer system that includes a graphical user interface and/or an Internet browser. To provide for interaction with a user, some implementations may be implemented on a computer having a display device, such as an LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from a source to a receiver over a dedicated physical link (e.g., fiber optic link, infrared link, ultrasonic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, and the computers and networks forming the Internet. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 3.0, FireWire, ATA/IDE, RS-232, RS-422, RS-485, 802.11 a/b/g/n, Wi-Fi, Wi-Fi-Direct, Li-Fi, Bluetooth, Ethernet, IrDA, FDDI (fiber distributed data interface), token-ring networks, or multiplexing techniques based on frequency, time, or code division. Some implementations may optionally incorporate features such as error checking and correction (ECC) for data integrity, or security measures, such as encryption (e.g., WEP), two factor authentication, and password protection.

In various embodiments, a computer system may include non-transitory memory. The memory may be connected to the one or more processors may be configured for encoding data and computer readable instructions, including processor executable program instructions. The data and computer readable instructions may be accessible to the one or more processors. The processor executable program instructions, when executed by the one or more processors, may cause the one or more processors to perform various operations.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

A number of implementations have been described. Nevertheless, it will be understood that various modification may be made. For example, advantageous results may be achieved if the steps of the disclosed techniques were performed in a different sequence, or if components of the disclosed systems were combined in a different manner, or if the components were supplemented with other components. Accordingly, other implementations are contemplated.

1. An apparatus to correlate data streams, the apparatus comprising:
a data stream validation engine configured to:
(i) receive a first data stream 120 comprising a stream of validated data transmitted via a first data link from a remote medical device 115 adapted to generate the first data stream in response to input signals received via a monitor link 110 connected to a patient to be monitored, wherein the medical device is configured to include in the first data stream a first authentication information associated with the patient;
(ii) receive a second data stream 130 comprising a stream of unvalidated data transmitted via a second data link from a remote communication device 125 adapted to generate the second data stream in response to user input information received from the patient, wherein the communication device is configured to include in the second data stream a second authentication information associated with the patient;
(iii) verify that the received first data stream and the second data stream are associated with the patient based on the first and second authentication information; and,
(iv) upon verification that the first and second data streams are associated with the patient, generate a first correlated data for transmission to a validated medical server configured to receive validated data streams of information about the patient,
wherein the medical device further comprises one or more validated medical devices, and the communication device further comprises one or more communication devices.

2. The apparatus of claim 1, wherein at least a portion of the second data link 130 comprises at least one wireless connection between the one or more communication devices and the data stream validation engine 140.

3. The apparatus of claim 1, wherein the one or more communication devices comprise at least one personal communication device.

4. The apparatus of claim 3, wherein the at least one personal communication device comprises a mobile phone.

5. The apparatus of claim 4, wherein the at least one personal communication device is further configured to operate a data pass through application to generate the second data stream by transmitting the user input information received from the patient without alteration.

6. The apparatus of claim 1, wherein the monitor link is configured to operatively couple to a pH sensor to monitor the acidity of a patient's esophagus in the course of diagnosing and treating gastroesophageal reflux disease (GERD).

7. The apparatus of claim 1, wherein the first and second authentication information comprise time stamp information.

8. The apparatus of claim 7, wherein the first and second authentication information further comprise rolling code information.

9. The apparatus of claim 8, wherein the first authentication information further comprises at least one member of the group consisting of hardware identification of the at least one communication device, hardware identification of the medical device, and a unique ID associated with the patient.

10. The apparatus of claim 1, further comprising an alarm system coupled to the data stream validation engine, wherein upon verification that the first and second data streams are not associated with the patient, generate an alarm message for transmission.

11. An apparatus to correlate data streams, the apparatus comprising:
a data stream validation engine configured to:
(i) receive a first data stream 120 comprising a stream of validated data transmitted via a first data link from a remote medical device 115 adapted to generate the first data stream in response to input signals received via a monitor link 110 connected to a patient to be monitored, wherein the medical device is configured to include in the first data stream a first authentication information associated with the patient;
(ii) receive a second data stream 130 comprising a stream of unvalidated data transmitted via a second data link from a remote communication device 125 adapted to generate the second data stream in response to user input information received from the patient, wherein the communication device is configured to include in the second data stream a second authentication information associated with the patient;
(iii) verify that the received first data stream and the second data stream are associated with the patient based on the first and second authentication information; and,
(iv) upon verification that the first and second data streams are associated with the patient, generate a first correlated data for transmission to a validated medical server configured to receive validated data streams of information about the patient.

12. The apparatus of claim 11, wherein at least a portion of the second data link 130 comprises at least one wireless connection between the communication device and the data stream validation engine 140.

13. The apparatus of claim 11, wherein the remote communication device is further configured to operate a data pass through application to generate the first data stream by transmitting without alteration the stream of validated data received via the first data link from the remote medical device.

14. The apparatus of claim 11, wherein the monitor link is configured to operatively couple to a pH sensor to monitor the acidity of a patient's esophagus in the course of diagnosing and treating gastroesophageal reflux disease (GERD).

15. The apparatus of claim 11, wherein the first and second authentication information comprise time stamp information.

16. The apparatus of claim 15, wherein the first and second authentication information further comprise rolling code information.

17. The apparatus of claim 16, wherein the first authentication information further comprises at least one member of the group consisting of hardware identification of the at least one communication device, hardware identification of the medical device, and a unique ID associated with the patient.

18. The apparatus of claim 11, further comprising an alarm system coupled to the data stream validation engine, wherein upon verification that the first and second data streams are not associated with the patient, generate an alarm message for transmission.

19. A method to correlate data streams, the method comprising:
providing a data stream validation engine configured to:
(i) receive a first data stream 120 through a remote communication device configured to operate a data pass through application, the first data stream comprising a stream of validated data transmitted via a first data link from a remote medical device 115 adapted to generate the first data stream in response to input signals received via a monitor link 110 connected to a patient to be monitored, wherein the medical device is configured to include in the first data stream a first authentication information associated with the patient;
(ii) receive a second data stream 130 comprising a stream of unvalidated data transmitted via a second data link from the remote communication device 125 adapted to generate the second data stream in response to user input information received from the patient, wherein the remote communication device is configured to include in the second data stream a second authentication information associated with the patient;
(iii) verify that the received first data stream and the second data stream are associated with the patient based on the first and second authentication information; and,
(iv) upon verification that the first and second data streams are associated with the patient, generate a first correlated data for transmission to a validated medical server configured to receive validated data streams of information about the patient.

20. The method of claim 19, wherein the first and second authentication information comprise time stamp information.
